(19) United States
(12) Patent Application Publication
(10) Pub. No.: US 2019/0080791 A1
(43) Pub. Date: Mar. 14, 2019

(54) SYSTEM AND METHOD OF MEDICATION DELIVERY AND ADHERENCE TRACKING

(71) Applicants: Collin Wolf, Hackettstown, NJ (US); Edwin Mellett, Pickens, SC (US); Chris Corsi, West Deptford, NJ (US)

(72) Inventors: Collin Wolf, Hackettstown, NJ (US); Edwin Mellett, Pickens, SC (US); Chris Corsi, West Deptford, NJ (US)

(21) Appl. No.: 16/127,369

(22) Filed: Sep. 11, 2018

Related U.S. Application Data
(60) Provisional application No. 62/557,948, filed on Sep. 13, 2017.
(60) Provisional application No. 62/640,644, filed on Mar. 9, 2018.

(51) Int. Cl. G16H 20/13; A61B 5/00; G16H 80/00; G06F 21/32

(52) U.S. Cl. CPC G16H 20/13; G06F 21/32; G16H 80/00; A61B 5/0022

(57) ABSTRACT

A medication delivery and adherence system, comprises a housing configured to mechanically connect to a portable computing and communication device, a controller disposed within the housing and communicatively connected to the portable computing and communication device, at least one removable sleeve disposed within the housing, the at least one sleeve configured to hold at least one dose of a medication, and at least one electromechanical actuator electrically connected to the controller, the electromechanical actuator configured to dispense the at least one dose of medication from the at least one sleeve. A method of verifying patient adherence to a treatment regimen is also described.

### CROSS-REFERENCE TO RELATED APPLICATIONS

This application claims priority to U.S. provisional patent application No. 62/557,948, filed Sep. 13, 2017, and U.S. provisional patent application No. 62/640,644, filed Mar. 9, 2018, both of which are incorporated herein by reference in their entirety.

### BACKGROUND OF THE INVENTION

Patient compliance to treatment regimens is a problem throughout the medical field, particularly in clinical and pharmaceutical trials, and in drug therapy settings where non-compliance is associated with significant negative clinical outcomes. Research demonstrates that a significant number of enrolled patients often fail to complete a clinical drug trial, making it difficult to accurately determine the clinical significance of intervention. Particularly in pharmaceutical trials, logistical and cost limitations can mean that clinicians have little to no direct interaction with patients during the trial, suggesting that clinicians often have inadequate means to ensure that patients are taking their medication on time, and often lack a significant means of intervention.

The problem of non-adherence can compound, because lower adherence rates can contribute to larger trial patient populations. To compensate for non-adherence in trials, often trials will enroll additional patients to derive statistical significance. Sometimes, a trial will need to be extended due to non-compliant participants, contributing to larger costs and missed market opportunities. Most importantly, the patient population will not achieve desired clinical outcomes when new therapies struggle to enter the market. Additionally, public health problems such as increased drug resistance, increased disease spread, and decreased patient outcomes are directly attributed to patient non-adherence. Also, problems such as increased drug-abuse have also been related to the high degree of un-checked accessibility of medications in the home.

Currently available methods of monitoring and encouraging patient adherence include “med-counts”, wherein the clinician periodically counts the pills left in the container to ensure that the proper number of pills are missing. Simple counting is problematic because it is easily faked. Another known method is the use of a “med journal”, wherein the patient is required to record the dates and times that they take their medications in a paper journal. Med journals add more responsibility to patients instead of easing their burden, because they require patients to remember to manually record adherence. Moreover, med journals do nothing to improve two-way patient/clinician communication, and can be altered after the fact.

Additionally, In the specialty pharmacy setting where high value drugs are delivered to patient populations, a large emphasis is placed on the monitoring of patient compliance by insurance companies and drug manufacturers to justify the high cost of the medication. This requires the specialty pharmacy to provide additional monitoring services and reporting procedures which increase the cost of care to patients. Often times these services are manual, requiring frequent touchpoints with the patient population through specialty pharmacy staff.

There is a need in the art for a system and method of medication delivery and tracking that reduces patient responsibility, allows clinicians to monitor patient adherence, delivers motivational support and allows clinicians or care-givers to intervene if needed. The present invention satisfies this need.

### SUMMARY OF THE INVENTION

In one aspect, a medication delivery and adherence system comprises a housing configured to mechanically connect to a portable computing and communication device, a controller disposed within the housing and communicatively connected to the portable computing and communication device, at least one removable sleeve disposed within the housing, the at least one sleeve configured to hold at least one dose of a medication, and at least one electromechanical actuator electrically connected to the controller, the electromechanical actuator configured to dispense the at least one dose of medication from the at least one sleeve. In one embodiment, the at least one removable sleeve is equipped with an identifier, and the housing comprises an interface configured to read the identifier. In one embodiment, the electromechanical actuator comprises a motor. In one embodiment, the electromechanical actuator further comprises a rotating barrel having a cavity with an outlet on one side of the barrel, wherein the cavity is large enough for one dose of medication to fit completely inside. In one embodiment, the system further comprises at least one spring positioned in the at least one sleeve.

In one embodiment, the system further comprises a non-volatile computer-readable medium comprising a set of instructions executed by a processor, the instructions comprising the steps of integrating a regimen received from a healthcare provider, identifying a patient, administering a dose of a medication to the patient according to the regimen, confirming that the dose has been administered, and sending updates to the healthcare provider when the dose has been administered. The medication delivery and adherence system of claim 6, wherein the instructions further comprise the step of notifying at least one individual not affiliated with the healthcare provider when the dose has been administered. In one embodiment, the instructions further comprise the step of delivering a different specific message or video to the patient after the dose has been administered or missed. In one embodiment, the message is configured manually by the clinician. In one embodiment, the message is configured automatically based on the regimen. In one embodiment, the instructions further comprise the step of displaying an instructional video related to the patient's adherence. In one embodiment, the instructions further comprise the steps of determining the patient's location, and changing the regimen based on the patient's location. In one embodiment, if the patient is driving, the regimen is changed to delay the dose until after the patient is no longer driving. In one embodiment, if the patient is at a restaurant, the regimen is changed to move forward the dose of a medication that must be taken with food.

In one embodiment, the instructions further comprise the steps of prompting the patient for written feedback, and transmitting the written feedback to the healthcare provider. In one embodiment, the instructions further comprise the steps of confirming that the dose has been taken, and transmitting information confirming that the dose has been taken. In one embodiment, the instructions further comprise the steps of measuring at least one physical or physiological parameter of the patient to confirm that the dose has been taken, and indicating that the dose has been taken. In one embodiment, the physical parameter is selected from the group consisting of an exhaled gas or vapor-species from the patient, the gas or vapor species selected from the group consisting of CO2, trace-gasses and alcohols. In one embodiment, the physiological parameter is selected from the group consisting of sweat composition, blood-vessel dilation, blood pressure, and temperature. The physiological or exhaled parameters may be measured by techniques such as optical spectroscopy, visual inspection or catalytic reaction sensors. In one embodiment, the instructions further comprise the steps of deciding via a probabilistic process whether to assign the patient to a control group, and administering a dose of a different medication to the patient if the patient is assigned to the control group.

In one embodiment, the system further comprises an authentication means, wherein the identifying step comprises comparing at least one authentication credential of the patient with a stored authentication credential in order to validate the identity of the patient. In one embodiment, the system further comprises a battery disposed within the housing. In one embodiment, the system further comprises a sensor selected from the group consisting of a temperature sensor and a humidity sensor, the sensor configured to measure the environment within or around the device. In one embodiment, the controller is configured to notify the patient or change the regime if the at least one medication has experienced an over-exposure to a damaging environmental condition. In one embodiment, the battery, the controller, and the electromechanical actuator are electrically connected to the portable computing device, and the battery is configured to provide power to the portable computing device. In one embodiment, the at least one medication comprises a co-medication. In one embodiment, the controller is communicatively connected to the portable computing device via a wireless connection. In one embodiment, the controller and the at least one electromechanical actuator are electrically connected to the portable computing device, such that the controller and the at least one electromechanical actuator are powered by a battery in the portable computing device. In one embodiment, the system further comprises a tamper detection means, configured to detect when the housing has been tampered with or when a dose of the medication has been removed outside of the programmed regime. In one embodiment, the system further comprises a tamper detection means, configured to detect when the housing has been tampered with, and wherein the instructions further comprise the step of notifying the healthcare provider when tampering has been detected.

In another aspect, a method of verifying patient adherence to a treatment regimen, comprises the steps of issuing a medication dispensing device containing at least one dose of a medication to a patient, enrolling the patient to the medication dispensing device via at least one identifying mechanism, setting a dosing schedule in the medication dispensing device, prompting the patient to dispense at least one dose of the medication according to the dosing schedule, verifying the identity of the patient, dispensing the at least one dose of the medication, confirming that the at least one dose of the medication was taken, and sending a confirmation message to a central database to verify that the patient took the dose of medication. In one embodiment, the identity of the patient is verified by checking at least one fingerprint of the patient. In one embodiment, the identity of the patient is verified by checking at least one biometric characteristic of the patient, selected from the group consisting of fingerprints, facial recognition, voice recognition, iris recognition, retinal identification, and DNA identification. In one embodiment, the identity of the patient is verified with a username and password. In one embodiment, the identity of the patient is verified using facial recognition. In one embodiment, the at least one dose of medication comprises at least one pill.

In one embodiment, the method further comprises the steps of setting a medication dispensing time window during which the at least one dose of medication should be taken, and, if the at least one dose of medication is not taken within the time window, sending an alert message to the central database to indicate that the patient did not take the at least one dose of the medication. In one embodiment, the medication dispensing device contains at least one dose of two different medications, and the dosing schedule includes entries for all doses of the two different medications. In one embodiment, the method further comprises the step of, after prompting the patient to dispense the at least one dose of the at least one medication, and if the patient elects not to dispense the at least one dose of the at least one medication, waiting for a predetermined time interval and then prompting the patient again to dispense the at least one dose of the at least one medication.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a schematic of one embodiment of a device of the invention;

FIG. 2 is a photograph of a prototype of a device of the invention;

FIG. 3A, FIG. 3B, and FIG. 3C are photographs of various views of a prototype of a device of the invention;

FIG. 4 is a photograph of a mockup of a device of the present invention;

FIG. 5 is a flow diagram of a user interface of the present invention;

FIG. 6 is a flow diagram of a user interface of the present invention;

FIG. 7 is a flow diagram of a user interface of the present invention;

FIG. 8 is a flow diagram of a user interface of the present invention;

FIG. 9 is a view of a clinician interface of the present invention;

FIG. 10 is a block diagram of a system of the present invention; and

FIG. 11 is a flow diagram of a method of the present invention.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smart phones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

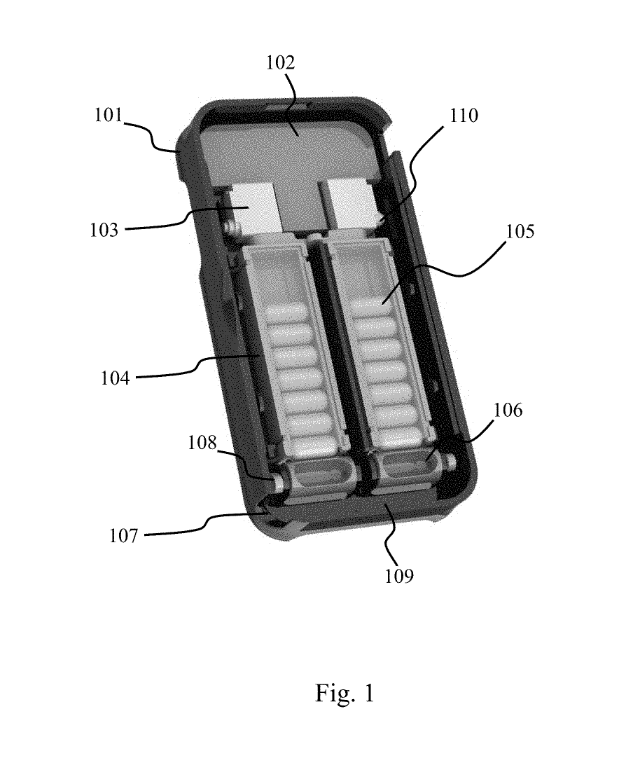

In one aspect, a system of the present invention comprises a medication dispensing device. In one embodiment, the medication dispensing device is built into a protective case for a portable computing device, for example a smart phone. In some embodiments, the portable computing device may be a portable computing and communication device. An exemplary embodiment of a medication dispensing device is shown in FIG. 1. A medication dispensing device comprises a housing 101 and one or more medication dispensing sleeves 104. Each sleeve 104 contains one or more doses of medication 105. In the exemplary embodiment of FIG. 1, the device includes two sleeves, but dispensing devices including one, three, four, or up to ten sleeves are envisioned. The sleeves 104 may further comprise one or more identification means, such that the controller 102 is capable of reading the identification means to validate which sleeves, and consequently which medications, are positioned in the device. Additionally, the sleeves and medications may be linked to a prescription and or assigned to a particular patient. Suitable identifiers include, but are not limited to, electrical circuitry delivering pre-programmed id codes, RFID, QR-code, linear barcodes, alphanumeric codes, or a system of extruded dots and/or pits along one or more sides of the sleeve. Identifiers of the present invention may be unique for each medication, i.e. all sleeves containing pill a include the same identifier, or may alternatively be unique for each sleeve, with no two sleeves have the same identifier.

A medication dispensing device of the present invention may be configured to dispense any suitable medication known in the art. In some embodiments, one or more sleeves of the medication dispensing device may contain a co-medication, including but not limited to an anti-drug. In some embodiments, one sleeve of the device contains a primary medication while another sleeve contains a co-medication for use with the primary medication. In some embodiments, one or more of the sleeves contains an anti-symptom drug, for example but not limited to an anti-overdose drug, an anti-nausea drug, an antihistamine drug, or any other suitable anti-drug known in the art.

The device further comprises a controller 102 and one or more electromechanical actuators 103 configured to dispense the medication doses 105 from the sleeves 104. In the exemplary embodiment, the actuators 103 are small servo motors, but the actuators could alternatively be any suitable method of controlling dispensation, including but not limited to different types of electric motors, solenoids, or any other suitable means for electrically-controlled, mechanically-actuated medication dispensation. The actuation system of the exemplary embodiment of FIG. 1 further comprises two actuation barrels 106, mechanically connected to the actuators 103 via gears 108 and 110 and a connecting member (not shown). Actuator 103 turns the gear 110, which in turn rotates gear 108. Each gear 108 is connected to an actuation barrel 106. The actuation barrels each comprise a hollow cavity with an opening on a lateral side of the barrel. As the barrel rotates, the cavity becomes fluidly connected to the inside of the corresponding medication sleeve 104, at which point the stack of medication doses 105 is pushed down toward the opening by a spring (not shown) or other mechanical actuation means, and one dose 105 is deposited into the cavity of one of the barrels 106. The barrel 106 then continues to rotate until the cavity is fluidly connected to a dispensing chamber 109, having an outlet 107. In some embodiments, the barrel 106 comprises a sensor, communicatively connected to the controller 102, for determining whether a medication dose is present in the cavity of the barrel. The sensor can verify that the medication dose has been pushed into the cavity when the cavity is fluidly connected with the medication sleeve, and also can verify that the medication dose has been properly released when the cavity is fluidly connected to the dispensing chamber. If the sensor detects that the medication has not properly been moved into or out of the cavity, in some embodiments the controller can return a signal to prompt the patient to shake or otherwise manipulate the medication dispensing device in order to properly dispense the dose. In one embodiment, the system further comprises one or more temperature or humidity sensors positioned within the device, configured to determine whether or not the medications within the device have been stored at appropriate temperature and humidity. In another embodiment, the system further includes one or more sensors configured to detect when the device has been compromised by tampering or damage. In such a situation, medications contained within the device may have been removed, or may no longer be safe for use. In one example, when tampering is detected, the system of the present invention notifies the clinician or other healthcare professional associated with the device. In some embodiments, a tamper detection system of the present invention may be specifically configured to detect whether a dose of medication has been removed from the device outside the programmed regime.

In some embodiments, controller 102 comprises one or more processing units, memory, and storage. The processing units may comprise one or more microcontrollers or systems-on-a-chip (SoCs). Where the actuators 103 require significant electrical current in order to operate, the controller 102 may additionally comprise one or more relays or transistors to operate the actuators 103. The controller 102 may further comprise one or more sensors, including but not limited to one or more optical sensors, fingerprint readers, accelerometers, linear or rotational position sensors, temperature sensors, humidity sensors, pressure sensors, or any other suitable sensors known in the art. In some embodiments, the one or more sensors may be configured to measure or monitor the medications within the device, whereas in other embodiments, additionally or alternatively, some or all of the one or more sensors may be configured to monitor the environment within or around the device. In some such embodiments, when one or more of the medications within the device have been exposed to a damaging environmental condition for more than a predetermined period of time, the controller may take a prescribed action, for example notifying the patient, notifying a clinician, or changing the medication regime to compensate for the detected environmental condition. In some embodiments, the storage of the controller comprises a set of stored instructions which, when executed in sequence by the one or more processing units, perform various steps of the invention. In some embodiments, the storage of the controller further includes dosing data related to the patient and the various medications, but in other embodiments this data is stored on an attached smart phone or on remote servers.

In some embodiments, the controller 102 further comprises an independent power source. The power source may optionally be a small battery meant to maintain system state in the event of intermittent power loss, or a larger battery or set of batteries configured to power the device independently of the portable computing device. In some embodiments, the battery or set of batteries is further configured to power the portable computing device itself, i.e. to act as a backup battery or charging source for the portable computing device. In some embodiments, the battery is a rechargeable lithium-ion or lithium polymer battery, but in other embodiments the battery is one or more user-replaceable alkaline cells. In some embodiments, the controller further comprises a Power Management Integrated Circuit (PMIC) for managing the battery charging and power distribution. The preceding examples of battery configurations are meant only as examples, and it is understood that any battery technology may be used. In some embodiments, the controller does not comprise a battery and instead draws power from the battery of the smart phone or other portable computing device to which it is attached.

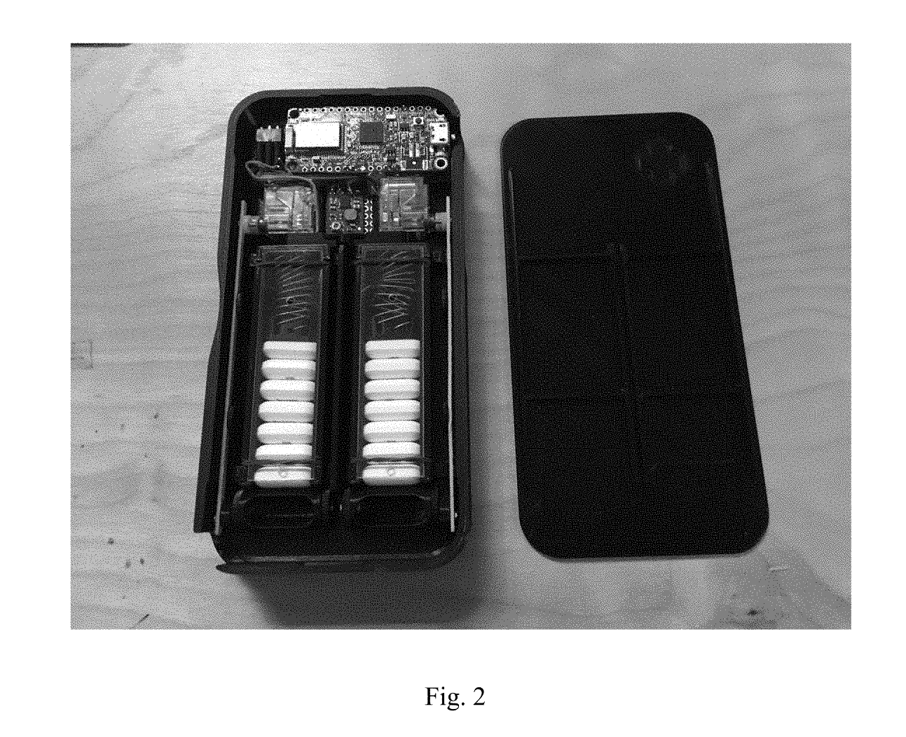

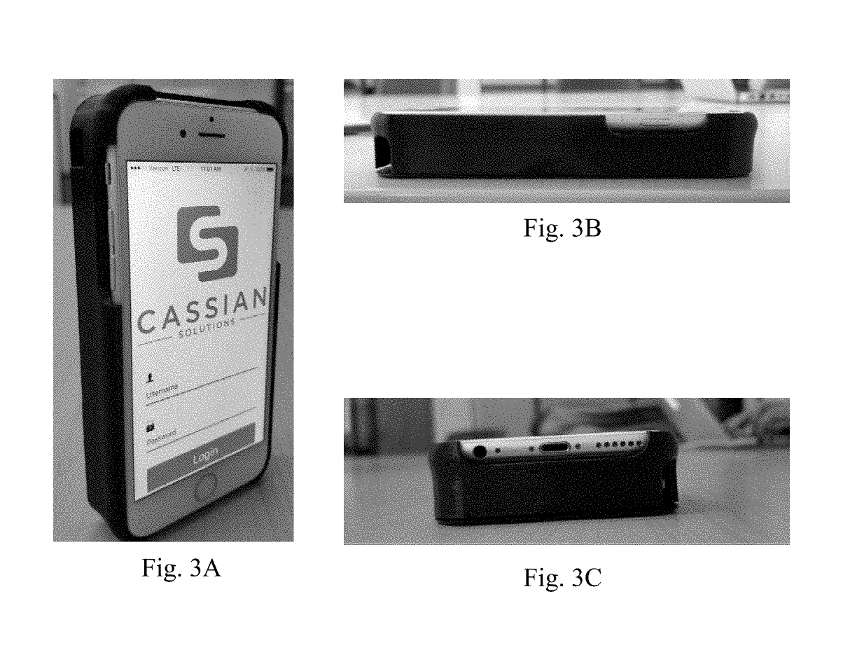

Referring now to FIG. 2, a photograph of a prototype based on the model of FIG. 1 is shown, with springs for actuating the medications in the sleeves. Alternate views of the prototype case, showing its size relative to an exemplary smart phone, are shown in FIG. 3A-FIG. 3C. FIG. 3A shows an isometric view of the prototype mounted to a smart phone, FIG. 3B shows a side view of the prototype, and FIG. 3C shows a bottom view of the prototype. As shown, in some embodiments, devices of the present invention include cutouts for access to buttons and charging ports.

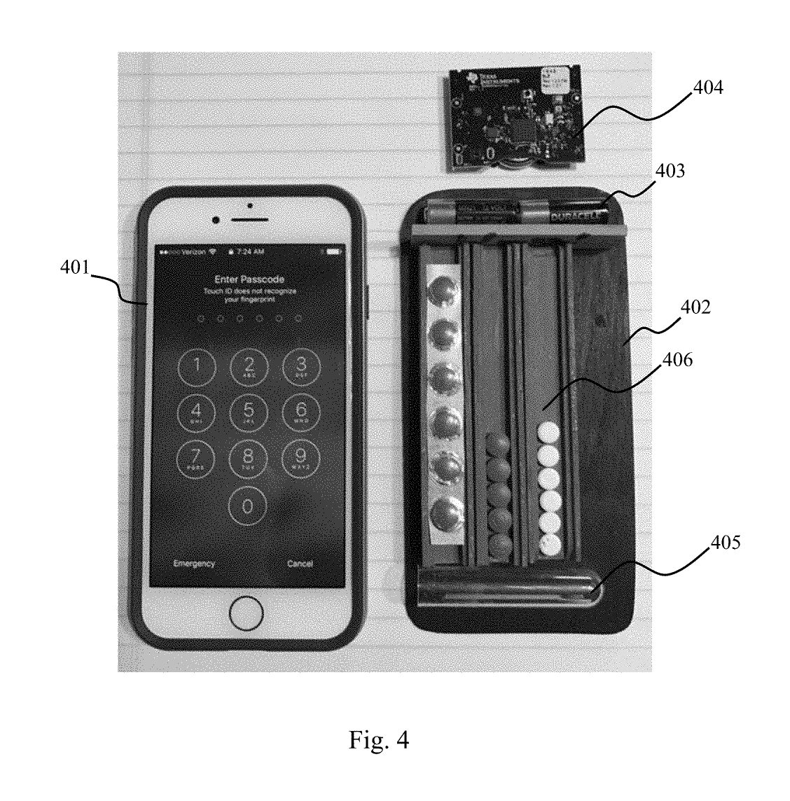

Referring now to FIG. 4, a mockup of one embodiment of a device of the present invention is shown. Next to an exemplary smart phone 401 is a plate 402 cut to scale, showing two batteries 403 and three medication sleeves 406. A microcontroller or SoC development board 404 is shown. At the bottom of the scale mock-up is a placeholder for a dispensing barrel 405. As shown in FIG. 4, the sleeves 406 may have different internal dimensions in order to accommodate medications of various sizes. In some embodiments, the sleeves will also have different outer dimensions, including a different width or depth, according to the requirements of the medications stored therein.

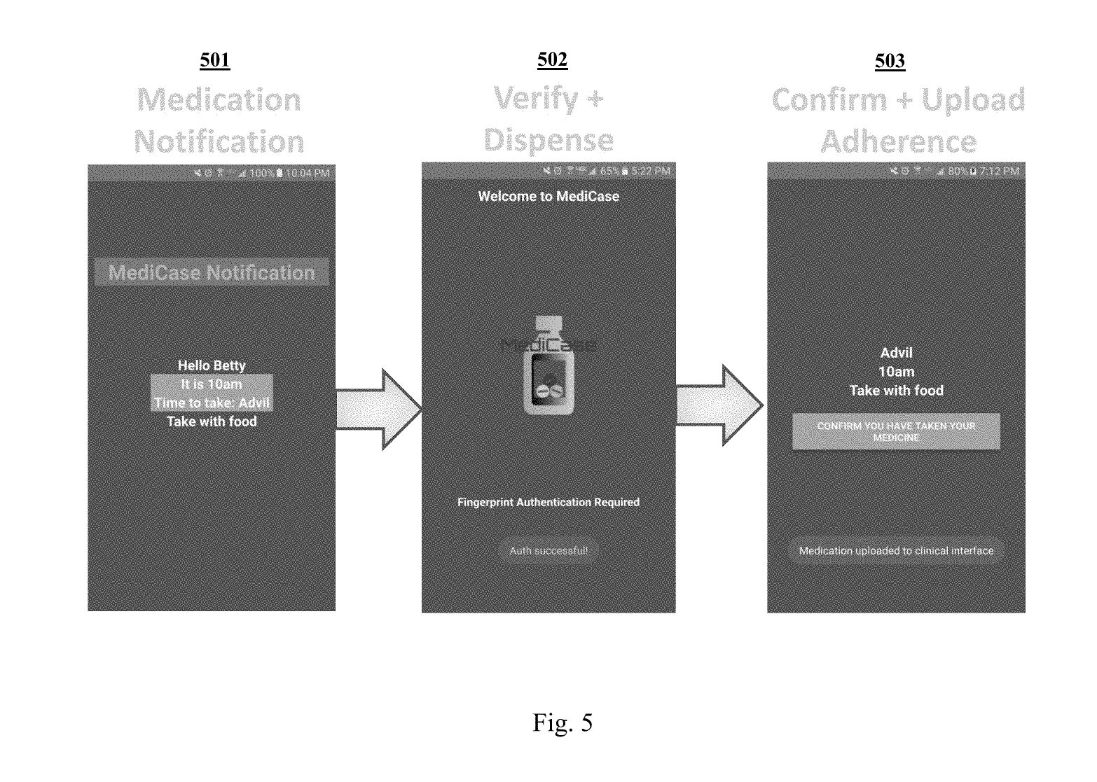

Some embodiments of the invention comprise a smart phone case and a smart phone application which together serve as a dispensing mechanism. One exemplary flow diagram of an application user interface of the present invention is shown in FIG. 5. In the workflow of FIG. 5, a user or a practitioner has set up a reminder to take Advil with food at 10 am. At the appropriate time, the smart phone application will present the user with a notification 501 that it is time to take Advil. As shown, the notification may also include supplemental instructions, for example to take the medication with food, or specific information such as when and how to properly take the specific medications. The notification 501 may be accompanied by a tactile signal such as vibration, an auditory signal such as a ringtone or notification beep, or a visual signal such as a notification light or blinking colors on the screen. In some embodiments, the notification is transmitted to the user or smart phone via some supplemental means, such as a text message or an e-mail. Notifications may further be introduced asynchronously via input by the clinician or another medical professional. In some embodiments, notifications may include videos related to the patient's adherence.

Once the notification is received, the smart phone application prompts the user to authenticate themselves to the app 502, in order to verify that the medication is being dispensed to the appropriate person. Authentication methods may include fingerprint authentication (as shown in the example of FIG. 5), a passcode, facial recognition, voice recognition, iris recognition, retinal identification, DNA identification, or any other suitable personal authentication means known in the art. Once the proper user has been authenticated, the application dispenses the appropriate medication. The fingerprint or other authentication information is then compared with a stored copy of the user's authentication information, the stored copy generated during an enrollment process which in some embodiments is supervised by the clinician. The application then prompts the user to confirm that they have taken their medication. In some embodiments, this confirmation is performed by the user pressing a button, as shown in the example 503. In other embodiments, the medication is confirmed via shooting a short video, for example of the user swallowing the pill or pills. In other embodiments, a photograph is uploaded to confirm that the medication was taken. In still further embodiments, the user confirms that the medication was taken by again swiping his or her fingerprint on a fingerprint scanner. Adherence may also alternatively or additionally be verified by measuring at least one physical or physiological parameter of the patient to confirm that the dose has been taken. For example, a device of the present invention may be configured to measure an exhaled gas or vapor-species from the patient, including but not limited to CO2, trace-gasses such as xenon, helium or argon, combinations of gasses, and/or alcohols. The device may further be configured to measure sweat composition, blood-vessel dilation, blood pressure, temperature, or any other measurable physiological parameter that may be used to confirm adherence with a dosing regime. The confirmation, along with any supplemental evidence, is then uploaded to a clinical interface in order to verify adherence.

In some embodiments, the system is configured to upload some or all of the data so that a clinician can see the data in real time. Over time, the server or the clinician application will accumulate data about the patient's medication habits and trends, from which statistics can be derived. If a patient is poorly compliant, the clinician can intervene to correct the treatment. The system may further be configured to notify other individuals besides the clinician when a dose is taken. For example, in one embodiment, other healthcare professionals may be notified as needed. In some embodiments, a patient's relatives and/or caregivers may be notified when a dose is taken. In some embodiments, some or all of the listed individuals may further be notified of other interactions between the patient and the system. In some embodiments, a notification to a caregiver or relative may include a non-specific message or telephone call, indicating that the patient might benefit from the caregiver's attention, or that intervention may be required. In this way, a system of the present invention can usefully communicate with caregivers, relatives, and third parties without divulging confidential medical information. In some embodiments, such caregiver notifications are generated based only on information recorded from the portable computing device or smart phone, and not from the medication delivery device itself.

In some embodiments, a system or method of the present invention may include blockchain encryption. For example, a method of the present invention may comprise the steps of blockchain encrypting some or all of the sensor data, prescription data, patient adherence data and/or clinical information. The blockchain encrypted data may be stored on a device of the present invention or may alternatively be stored in a computing cloud. In some embodiments, some or all of the blockchain encrypted data related to one device of the present invention may be stored on one or more remote central servers, or may alternatively be stored on other devices of the present invention.

The system may further be configured to generate positive reinforcements for the patient following favorable interactions with the device. Positive reinforcements or other key medical information provided by systems of the present invention may in some embodiments be inserted by the clinician or other healthcare professional, or may alternatively be automatically configured based on the nature of the clinical trial or trial duration. Such notifications and reinforcements may be sent to the patient using SMS text message or in-app notifications where applicable. In this way, devices of the present invention may further encourage the user to make use of the devices regularly at scheduled intervals. Although notifications are typically in one direction either from clinician to patient or vice versa, some notifications of the present invention may include prompts, for example feedback prompts for the patients. In one example, a patient is periodically prompted by a system of the present invention to provide feedback to the clinician on the medication or medications they have been taking, or the nature of the clinical trial as a whole. In some embodiments, notifications of the present invention may comprise delivering a different specific message or video to the patient after a dose has been administered or missed, for example a positive reinforcement message after a dose has been administered, or a reminder after a dose has been missed.

Systems of the present invention may further autonomously modify one or more dosing schedules for a patient based on the patient's location. For example, if a medication is best taken with food, systems of the present invention may use one or more sensors, for example a GPS receiver, to determine that a patient is currently in a restaurant, and most likely eating dinner. The system may then modify the dosing schedule and notify the patient to take one or more medications earlier or later than originally scheduled. In another embodiment, a medication is best taken on an empty stomach, and a system may postpone a scheduled dose for a few hours after a patient is detected to be eating. Autonomous modifications to dosing schedules may be enabled, disabled, or augmented manually by the clinician or other healthcare professional. In another example, a medication may impact a patient's ability to operate a vehicle, operate heavy machinery, or perform work duties. In such an example, a system of the present invention may detect that a patient is moving at high speed, or located near a place of work. The system may then alert the patient to remind them of the dangers of performing such hazardous activities while taking the medication.

Although some embodiments of the present invention are described in a clinical setting, devices and methods of the present invention may further be used in non-clinical settings for maintaining a dosing routine. Devices of the present invention may be used for example to remind a user to take a multivitamin or other nutritional supplement at regular intervals. Devices and methods of the present invention may further be used to treat or cure addiction, for example opioid use disorder, by scheduling and verifying compliance with a treatment course of methadone.

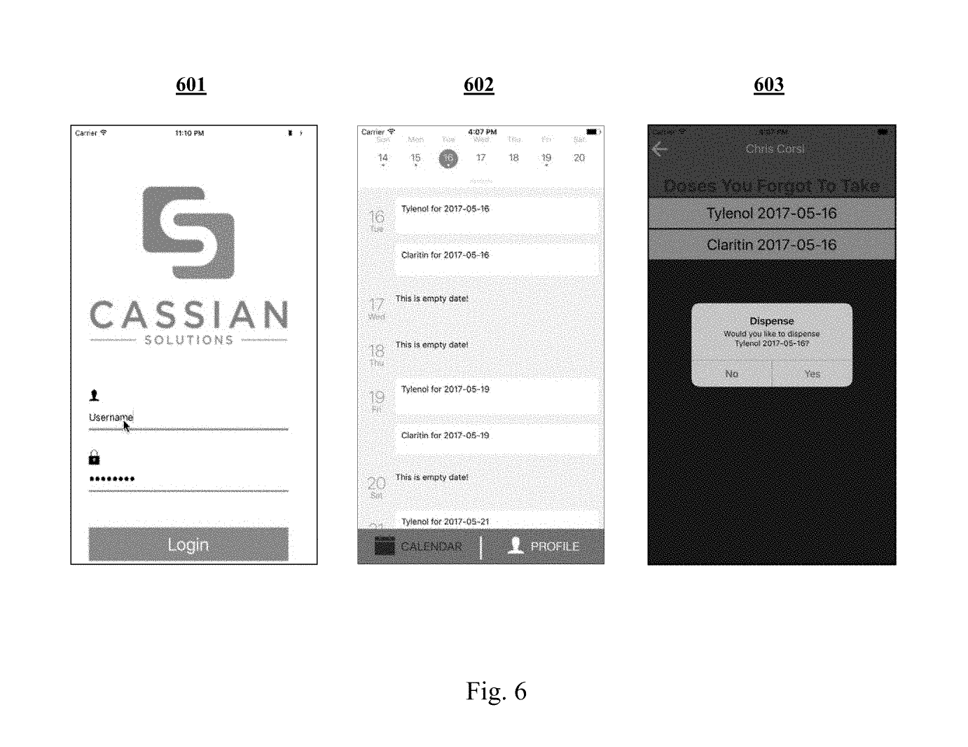

Additional screens of an alternate application user interface workflow are shown in FIG. 6. An alternative username/password based authentication screen 601 may be used in place of the fingerprint authentication system shown generally in FIG. 5. Some embodiments of an application of the present invention may include a screen displaying a calendar view of past or upcoming doses, shown in 602. Different medications may be shown as distinct calendar events, as shown in 602, or in some embodiments multiple different medications may be combined into a single calendar event, for example when a physician or clinician recommends that two or more medications be taken together. In some embodiments, the application comprises a calendar system with multiple capabilities, and medicines that have missed a dose can then be administered if acceptable as per a regimen outline. The app can notify patients when it is time to take their medication, and if the regimen permits, the patient can receive a dose that they have recently missed.

A further interface screen 603 shows a listing of the medications with doses missed. In one exemplary embodiment, a user forgets to take or neglects to take a dose at the predetermined time, at which point the dose is added to a list of missed doses to be taken later. In some embodiments, the clinician or physician can set different behaviors for a missed dose. For example, a physician might set a certain medication to skip a dose that was missed if too much time has elapsed since the intended dispensing time. In some embodiments, the dose missed generates an alert on a physician interface of the present invention. In some embodiments, the user application sends additional alerts to remind a patient to take a dose if the dose is not dispensed within a predetermined time window.

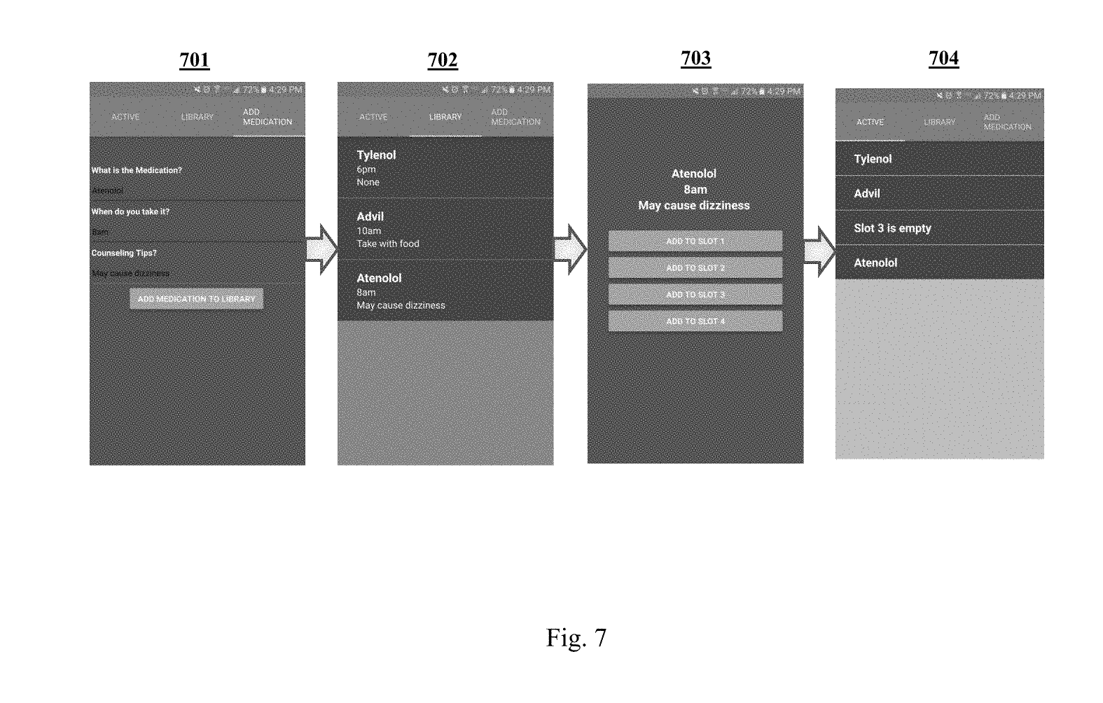

An exemplary physician or clinician user interface is shown in FIG. 7. The depicted screens are part of the interface used by the clinician to add medications to a patient's device and, in some embodiments, set the parameters for administering the medications. Interface screen 701 is an exemplary medication adding screen, allowing the physician or clinician to select the type of medication, set the timing for dosage, and additionally include any known side effects, in this example dizziness, to be displayed with the medication alert. Screen 702 shows the “library” view of the currently-loaded medications. From screen 702, the physician may select any medication to be taken back to the detail view in 701, to set the parameters of that medication.

Screen 703 allows the physician to add a medication sleeve to a particular slot in the back of the medication dispensing device. In some embodiments, a locking mechanism secures the back cover to the medication dispensing device, and is only released when the application is on a screen allowing for a medication sleeve to be added or removed from the medication dispensing device. Finally, screen 704 shows which medications are “active,” or currently loaded into slots and ready for dispensing. Using this device, a clinician may provide a patient with medication doses and directly insert them into the medication dispensing device, which relieves the patients of the responsibilities of keeping track of their medication bottle or counting pills themselves.

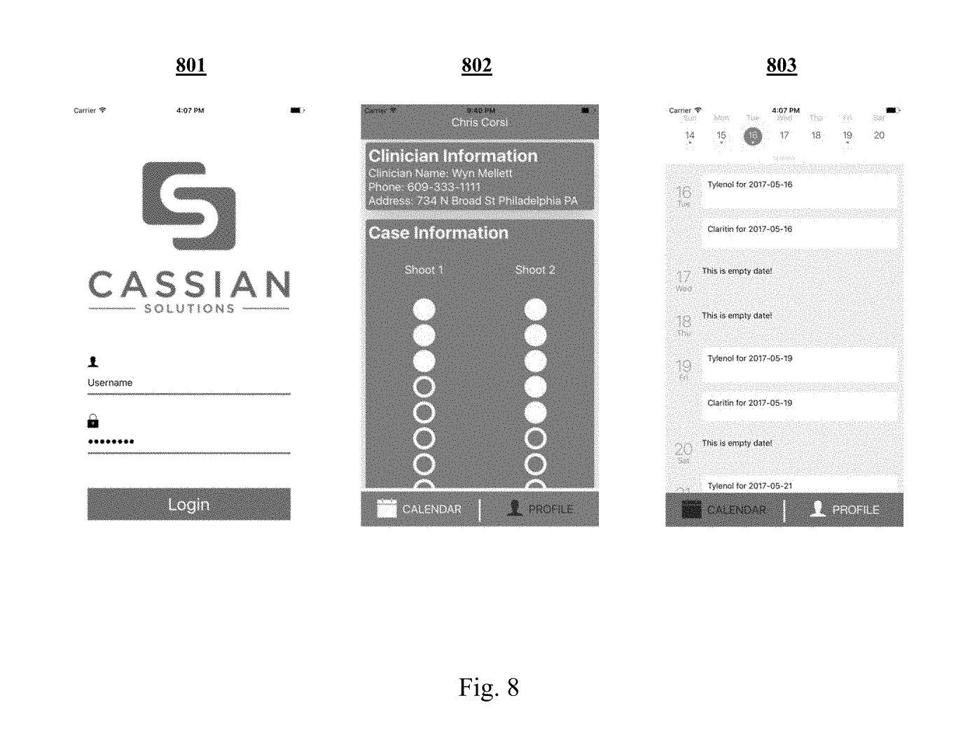

An alternate embodiment of the clinician/physician user interface is shown in FIG. 8. Interface screen 801 is identical to the interface screen 601 in the patient interface. In this embodiment, a single application is used to control and dispense the medications, where different users have access to different screens and interface elements depending on whether they authenticate as a patient, clinician, or physician. Supposing a clinician logs in with their credentials to screen 801, in some embodiments they will be presented with screen 802, which shows the patient's name and the name and contact information of the clinician on the top, and information about the currently loaded medications underneath. In this embodiment, screen 802 also displays how many doses remain in each of the two medication sleeves. In some embodiments, the smart phone application keeps track of the remaining doses by subtracting the doses dispensed from the total number of doses originally loaded. In other embodiments, the dispensing device includes one or more position or proximity sensors configured to determine or verify how many doses remain in each medication dispensing sleeve. Screen 803 shows a physician calendar view, similar to the patient calendar view shown in screen 602. From this screen, a clinician or physician can see what doses the patient has scheduled.

In some embodiments, investigators can enroll patients in a clinical trial using a system of the present invention. A patient may receive a medication dispensing device with medications stored within. The patient may then be directed to install an application on their smart phone. The application then integrates the regimen as an investigator provides the regimen through the clinical interface. The device application confirms doses administered, identifies the correct patient before administering each dose, collects patient data, and sends updates to the investigator. In some embodiments, some or all data is stored in a database or cloud storage in accordance with HIPAA guidelines. The clinical interface can then flag or identify therapy issues if the patient is partially adherent (missing doses) or neglecting to get refills. The clinical interface can then send a notification to the clinician. If necessary, the clinician can then engage the patient and improve the therapy.

In some embodiments, clinical trials of the present invention may be configured, for example by the clinician or another healthcare professional, to include a control group who is given a placebo in place of the medication. In some embodiments, the patients who are given the placebo are picked randomly by systems of the present invention, and that information may or may not be shared with the clinician or healthcare professional. Such a system may reinforce the accuracy of the clinical trial. Clinicians cannot be biased against patients in the control group if they do not know which patients are in the control group and which are not. In another embodiment, clinical trials of the present invention may involve a comparison of two competing medications, for example a known effective medication and an experimental medication. Similarly to the control group example described above, a system of the present invention may randomly select patients of the clinical trial to receive the known effective drug or the experimental alternative.

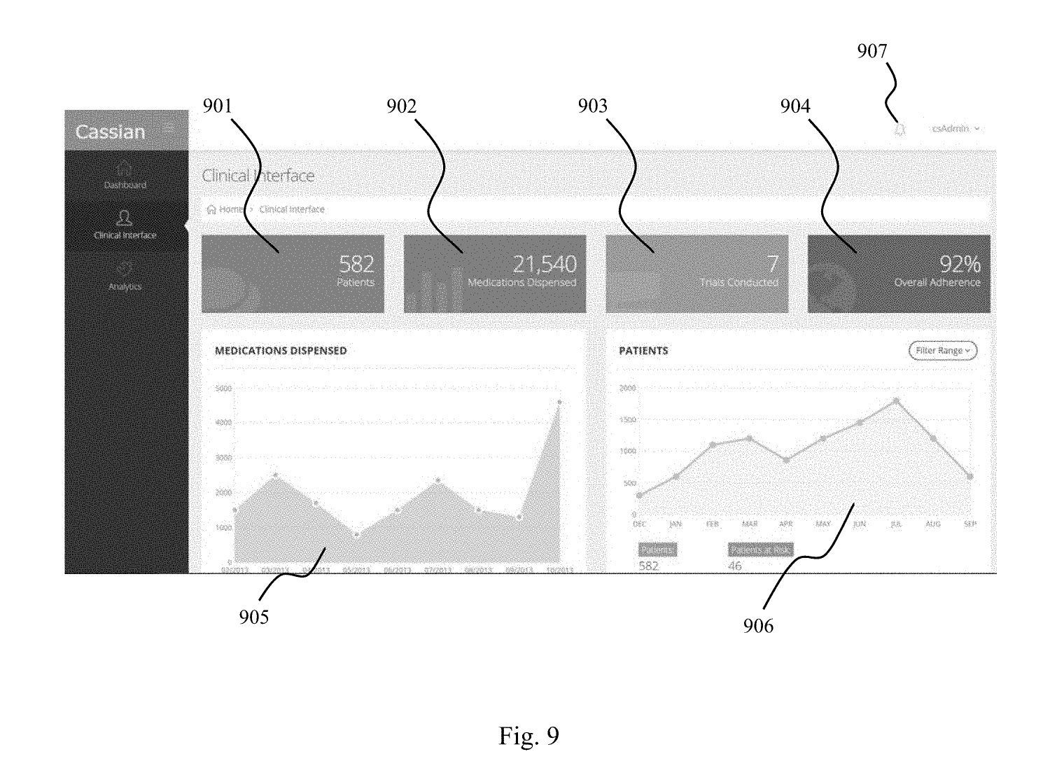

Some embodiments of the invention include a clinical user interface for monitoring compliance of a single patient or a group of patients participating in a study. An exemplary embodiment of such an interface is shown in FIG. 9. A clinical interface of the present invention may include one or more numerical indicators, for example but not limited to an indicator of the number of patients currently enrolled 901, the total number of medications dispensed 902, the total number of trials conducted 903, or the overall adherence percentage 904. In some embodiments, a clinical interface may also include graphical data, for example but not limited to time-series graphs of the medications dispensed per month 905, patients enrolled per month 906, or any other graphical data set that might be helpful to a clinician. In some embodiments, a clinical interface may also include a notification icon 907, which may change to indicate the presence of notifications or alerts, for example if a patient has missed a dose or if a patient has requested information from the clinician via the mobile application.

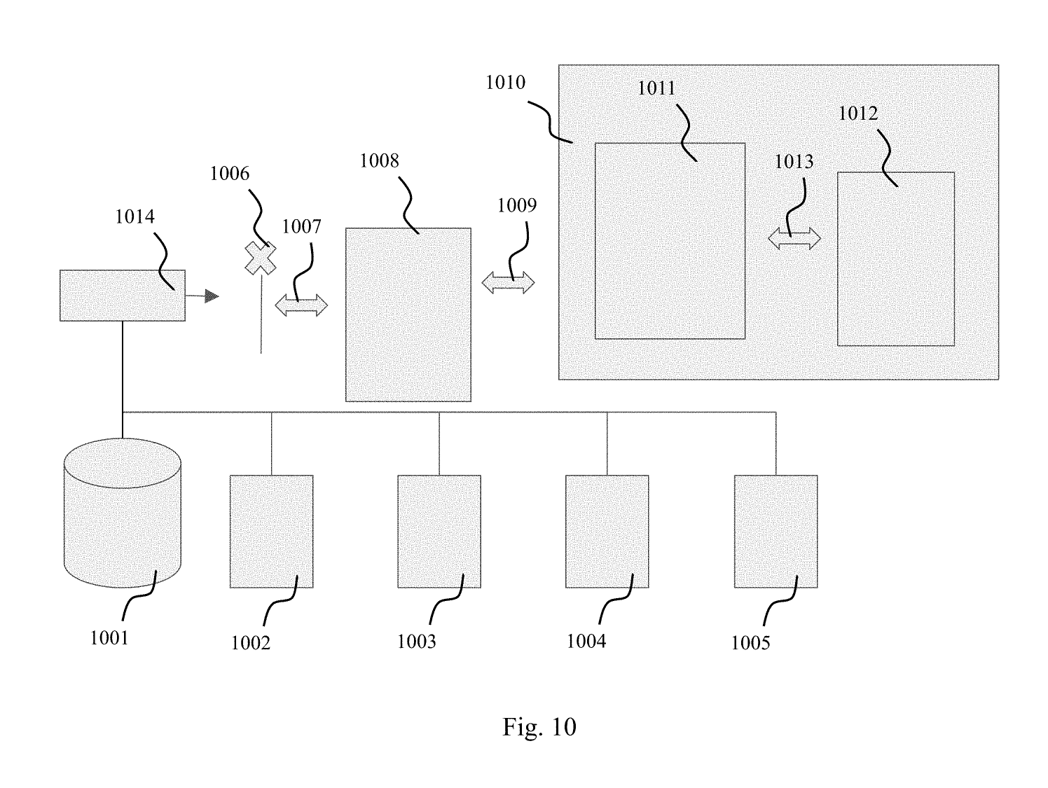

With reference now to FIG. 10, a block diagram of an exemplary embodiment of a system of the present invention is shown. The depicted embodiment of the system comprises a server application 1001 connected to the Internet 1014, along with several mobile, web-based, and installed applications including but not limited to a clinician application 1002, caregiver application 1003, primary care physician application 1004, and pharmacy application 1005. The system further comprises a smart phone 1008 belonging to or issued to a patient, which is connected to the Internet 1014 via the mobile network 1006 and cellular data connection 1007. The smart phone 1008 is also connected to the medication dispensing device 1010 via a Bluetooth connection 1009. The medication dispensing device 1010 comprises a controller 1011 and one or more medication dispensing sleeves 1012, electrically connected to one another via physical connection 1013.

As described herein, an exemplary smart phone 1008 may comprise a variety of sensors, display elements, and input devices, including but not limited to a CPU, a battery, a display, a keypad, a biometric identification system, Bluetooth, cellular, and/or wi-fi radios, an alarm timer, and one or more stored applications. A medication dispensing device controller 1011 of the present invention may comprise a set of elements including but not limited to a battery, a computing device, a cellular, Bluetooth, or wi-fi radios, an ID reader, a pen connector, a temperature sensor, a humidity sensor, a dispensing tray, a latch sensor, one or more medication dispensing sleeves, and a series of computing instructions stored on a non-volatile computer-readable storage. The one or more medication dispensing sleeves 1012 may each comprise one or more connectors, one or more medication doses, sometimes in the form of pills, an identifier, for example a unique ID, a security means, and a dispensing means.

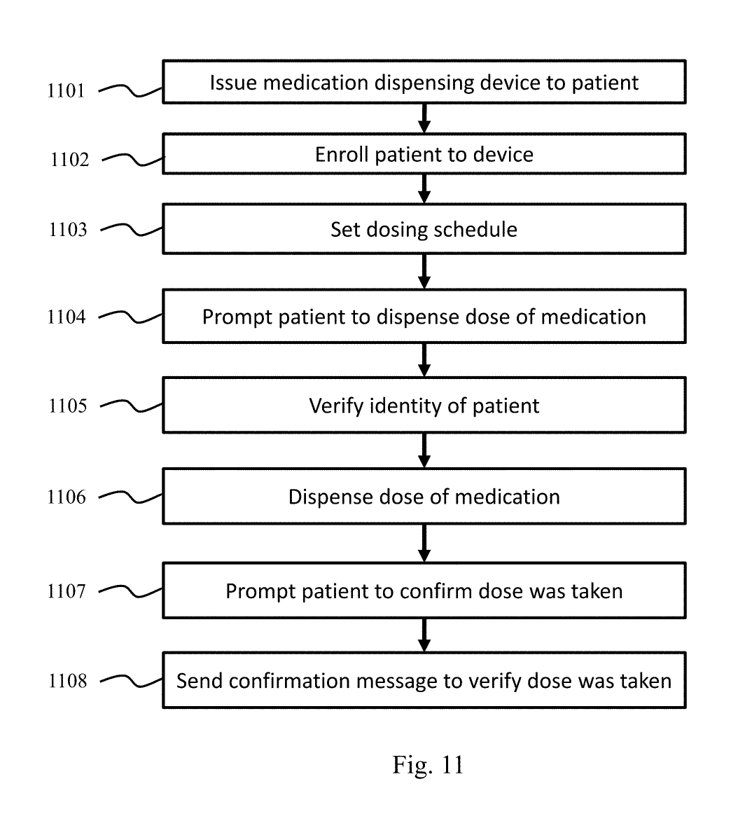

With reference to FIG. 11, a method of the present invention is shown. The method includes step 1101, issuing a medication dispensing device containing at least one dose of a medication to a patient; step 1102, enrolling the patient to the medication dispensing device via at least one identifying mechanism; step 1103, setting a dosing schedule in the medication dispensing device; step 1104, prompting the patient to dispense at least one dose of the medication according to the dosing schedule; step 1105, verifying the identity of the patient; step 1106 dispensing the at least one dose of the medication; step 1107, prompting the patient to confirm that the at least one dose of the medication was taken; and step 1108, sending a confirmation message to a central database to verify that the patient took the dose of medication.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A medication delivery and adherence system, comprising:
a housing configured to mechanically connect to a portable computing and communication device;
a controller disposed within the housing and communicatively connected to the portable computing and communication device;
at least one removable sleeve disposed within the housing, the at least one sleeve configured to hold at least one dose of at least one medication; and
at least one electromechanical actuator electrically connected to the controller, the electromechanical actuator configured to dispense the at least one dose of medication from the at least one sleeve.

2. The medication delivery and adherence system of claim 1, wherein the at least one removable sleeve is equipped with an identifier; and
wherein the housing comprises an interface configured to read the identifier.

3. (canceled)

4. The medication delivery and adherence system of claim 1, wherein the electromechanical actuator further comprises a rotating barrel having a cavity with an outlet on one side of the barrel, wherein the cavity is large enough for one dose of medication to fit completely inside.

5. (canceled)

6. The medication delivery and adherence system of claim 1, further comprising a non-volatile computer-readable medium with a set of instructions stored thereon, that when executed by a processor, perform the steps of:
integrating a regimen received from a healthcare provider;
identifying a patient;
administering a dose of a medication to the patient according to the regimen;
confirming that the dose has been administered; and
sending updates to the healthcare provider when the dose has been administered.

7. (canceled)

8. The medication delivery and adherence system of claim 6, wherein the instructions further comprise the step of notifying a caregiver that attention may benefit the patient, without revealing confidential medical information.

9. The medication delivery and adherence system of claim 6, wherein the instructions further comprise the step of notifying a caregiver that intervention may be required based only on activity reported from the portable computing and communication device and not from the medication delivery and adherence system.

10. The medication delivery and adherence system of claim 6, wherein the instructions further comprise the step of delivering a different specific message or video to the patient after the dose has been administered or missed.

11-13. (canceled)

14. The medication delivery and adherence system of claim 6, wherein the instructions further comprise the steps of:
determining the patient's location; and
changing the regimen based on the patient's location.

15-17. (canceled)

18. The medication delivery and adherence system of claim 6, wherein the instructions further comprise the steps of:
confirming that the dose has been taken; and
transmitting information confirming that the dose has been taken.

19. The medication delivery and adherence system of claim 6, wherein the instructions further comprise the steps of:
measuring at least one physical or physiological parameter of the patient to confirm that the dose has been taken; and
indicating that the dose has been taken.

20. The medication delivery and adherence system of claim 19, wherein the physical parameter is selected from the group consisting of an exhaled gas or vapor-species from the patient, the gas or vapor species selected from the group consisting of CO2, trace-gasses and alcohols.

21. The medication delivery and adherence system of claim 19, wherein the physiological parameter is selected from the group consisting of sweat composition, blood-vessel dilation, blood pressure, and temperature.

22. The medication delivery and adherence system of claim 6, wherein the instructions further comprise the steps of:
deciding via a probabilistic process whether to assign the patient to a control group; and
administering a dose of a different medication to the patient if the patient is assigned to the control group.

23. The medication delivery and adherence system of claim 6, further comprising an authentication means, wherein the identifying step comprises comparing at least one authentication credential of the patient with a stored authentication credential in order to validate the identity of the patient.

24. The medication delivery and adherence system of claim 6, wherein the instructions further comprise the step of blockchain encrypting at least one element of recorded data.

25. (canceled)

26. The medication delivery and adherence system of claim 1, further comprising a sensor selected from the group consisting of a temperature sensor and a humidity sensor, the sensor configured to measure the environment within or around the device.

27. The medication delivery and adherence system of claim 26, wherein the controller is configured to notify the patient or change the regime if the at least one medication has experienced an over-exposure to a damaging environmental condition.

28. (canceled)

29. The medication delivery and adherence system of claim 1, wherein the at least one medication comprises a co-medication.

30. The medication delivery and adherence system of claim 1, wherein the controller is communicatively connected to the portable computing device via a wireless connection.

31. The medication delivery and adherence system of claim 1, wherein the controller and the at least one electromechanical actuator are electrically connected to the portable computing device, such that the controller and the at least one electromechanical actuator are powered by a battery in the portable computing device.

32. The medication delivery and adherence system of claim 1, further comprising a tamper detection means, configured to detect when the housing has been tampered with or when a dose of the medication has been removed outside of the programmed regime.

33. (canceled)

34. A method of verifying patient adherence to a treatment regimen, comprising the steps of:
issuing a medication dispensing device containing at least one dose of a medication to a patient;
enrolling the patient to the medication dispensing device via at least one identifying mechanism;
setting a dosing schedule in the medication dispensing device;
prompting the patient to dispense at least one dose of the medication according to the dosing schedule;
verifying the identity of the patient;
dispensing the at least one dose of the medication;
confirming that the at least one dose of the medication was taken; and
sending a confirmation message to a central database to verify that the patient took the dose of medication.

35. The method of claim 34, wherein the identity of the patient is verified by checking at least one biometric characteristic of the patient, selected from the group consisting of fingerprints, facial recognition, voice recognition, iris recognition, retinal identification, and DNA identification.

36-37. (canceled)

38. The method of claim 34, further comprising the steps of:
setting a medication dispensing time window during which the at least one dose of medication should be taken; and
if the at least one dose of medication is not taken within the time window, sending an alert message to the central database to indicate that the patient did not take the at least one dose of the medication.

39. The method of claim 34, wherein the medication dispensing device contains at least one dose of a plurality of different medications, and the dosing schedule includes entries for all doses of the different medications.

40. The method of claim 34, further comprising the step of, after prompting the patient to dispense the at least one dose of the at least one medication, and if the patient elects not to dispense the at least one dose of the at least one medication, waiting for a predetermined time interval and then prompting the patient again to dispense the at least one dose of the at least one medication.

41. The medication delivery and adherence system of claim 6, wherein the instructions further comprise the steps of:
determining deviations from a past regimen; and
changing the regimen based on the determined deviations.
